(19) United States
(12) Patent Application Publication
(10) Pub. No.: US 2026/0263492 A1
(43) Pub. Date: Sep. 10, 2026

(54) ENERGETIC PRODUCT BASED ON CARBOHYDRATES IN THE FORM OF SOLID GELATIN TO INCREASE PERFORMANCE DURING PHYSICAL OR SPORTS ACTIVITY

(71) Applicant: ENERVIT S.P.A., Milano (IT)

(72) Inventors: Paolo Petroni, Giussano (IT); Diego Musazzi, Legnano (IT)

(73) Assignee: ENERVIT S.P.A., Milano (IT)

(21) Appl. No.: 18/846,988

(22) PCT Filed: Mar. 14, 2023
(86) PCT No.: PCT/IB2023/052470

§ 371 Date: Sep. 13, 2024

(30) Foreign Application Priority Data
Mar. 14, 2022 (IT) 102022000004889

(51) Int. Cl. A61K 31/7004; A61K 9/06; A61K 31/122; A61K 31/51; A61K 33/00; A61K 33/06; A61K 47/36; A61K 47/42

(52) U.S. Cl. CPC A61K 31/7004; A61K 9/06; A61K 31/122; A61K 31/51; A61K 33/00; A61K 33/06; A61K 47/36; A61K 47/42

(57) ABSTRACT

The invention concerns an energetic product based on carbohydrates in the form of highly palatable and digestible solid gelatin containing monosaccharides and a disaccharide that is protein and fat free to be taken during an intense physical effort or during the performance of a sports activity to provide the body with readily available carbohydrates and increase or maintain sports performance.

### FIELD OF THE INVENTION

The present invention concerns an energetic product based on carbohydrates in the form of solid gelatin and its use to improve physical performance of the human body during sports activity.

The present invention originates in the field of food and dietary supplements and nutritional products.

In particular, the present invention concerns a colloidal product in solid form with a high carbohydrate content to be taken during an intense physical effort and/or during the performance of a sports activity to provide the body with readily available carbohydrates and increase or maintain sports performance.

### BACKGROUND ART

The correlation existing between carbohydrate intake during sports practise and performance in the physical exercise has been known since the beginning of the last century and documented in the scientific literature for example by Krogh & Lindhard, 1920.

In 2019 Jeukendrup & Gleeson documented how the simple carbohydrates taken during sports activity act as an immediately oxidizable substrate and suitable to provide energy to the cells of the muscle tissue involved in the physical activity and to the cells of the central nervous system.

To make adequate doses of carbohydrates available during sports performance, various nutritional strategies have been proposed and adopted. Among these, the main ones envisage the intake of carbohydrates both before physical exercise to provide the human body with adequate concentrations of glycogen at the muscular and hepatic level and during the physical exercise in order to try and maintain blood glucose levels almost constant.

Typically these nutritional strategies are implemented by the athletes through the intake of products that provide carbohydrates. Generally these products are available in the form of ready-to-eat beverages, gels and bars.

The nutritional strategies and the food supplements based on carbohydrates intended for the subjects practising sports activity are aimed at building a nutritional regime that essentially responds to the following nutritional scheme:
intake of an amount of carbohydrates up to 60 g carbohydrates/hour in the case of activities lasting approx. 2 hours,
intake of an amount of carbohydrates up to 90 g carbohydrates/hour in the case of particularly intense and long physical efforts, typically exceeding 2.5/3 hours like in the course of trainings and sports competitions.

Starting from these well-established scientific bases, the European Food Safety Authority (EFSA) has recently proposed a claim regarding the intake of carbohydrate solutions during the physical exercise (EFSA, 2018).

The Commission itself has also highlighted the existence of a cause-effect relationship between consumption of carbohydrate solutions and improvement in physical performance during the high-intensity and long-lasting physical exercise and has therefore concluded: “Carbohydrate solutions can contribute to the improvement of physical performance during a high-intensity and long-lasting physical exercise in healthy trained adults”.

This conclusion is based on well-established scientific notions about carbohydrate metabolism summarized below.

Carbohydrates taken with the diet are hydrolysed at the gastro-intestinal level into their monomers (monosaccharides) and then absorbed by means of transport processes mediated by carrier proteins, which are found in the intestinal epithelial cells. The process of carbohydrate absorption involves the passage of nutrients from the intestinal lumen to the blood circulatory system by crossing two membranes: the luminal membrane and the contraluminal membrane of the intestinal epithelial cells. The carrier proteins which are specific for different saccharides are expressed on the luminal membrane of the epithelial cells.

For example, the carrier protein specific for glucose transport across the luminal membrane of the epithelial cells is known as sodium-dependent glucose transporter 1 (SGLT1), while the carrier protein specific for fructose is known as GLUT5.

Once the monosaccharides have entered the epithelial cells, a transport protein on the contraluminal membrane (GLUT2) then allows the transfer of the monosaccharides into the bloodstream.

Several scientific studies have suggested that in correspondence with the intake of high amounts of carbohydrates from the single glucidic source of glucose, the specific transport protein that promotes assimilation of that specific carbohydrate becomes saturated. After the saccharide-transporting carrier proteins are saturated, the further intake of that same saccharide does not result in increased assimilation at the intestinal level and consequently there is no increase in the oxidation rate. Under these conditions there are therefore no conditions to improve the athlete's physical performance beyond the limit made possible by the intake of 60 g per hour of glucose alone.

To overcome this problem, the athletes resort to the supplementation of specific carbohydrates during the physical exercise. These carbohydrates are contained in food supplements or foodstuffs that can be in liquid form of sugary solutions, in semi-solid form of gel or in solid form, for example energy bars.

The energetic products in liquid or gel form are the most widespread products and easy to use to take high amounts of carbohydrates during sports activity, especially motor activities such as cycling, endurance, running, marathon, triathlon.

The supplements in liquid and gel form have the advantage of being easy and fast to ingest even at high rates. In addition, their intake allows to have carbohydrates readily available to support even prolonged effort and to obtain an even very quickly gastric emptying.

However, the products in liquid or gel form have the drawback of having a low satiating power and not being satisfying for the athlete. These aspects play an important role in the dietary regimen of the athlete, especially when the athlete is engaged in intense and prolonged efforts beyond 3 hours.

It has been found that in sports activity, in particular in the aerobic activity in which physical activity is prolonged, such as cycling and endurance competitions, the athletes have a “psychological” need to chew solid products, to achieve adequate satisfaction and a sense of satiety, a factor that is also quite important from a nutritional point of view.

In light of these needs, it is felt the need to have food energetic products in solid form, typically nutritional bars.

However, taking supplements in solid form during prolonged physical activity, although it meets certain needs of the athlete, for example the capability of inducing a sense of satiety during physical activity, can present a number of drawbacks.

A first significant drawback is represented by the formulations of the supplements in solid form since, with the same carbohydrate content, they have a protein and fat content that cannot be technically eliminated, two categories of nutritional elements whose intake during activity presents contraindications from the nutritional and psychological point of view.

From the nutritional-metabolic point of view of the human body, the supply of proteins and fats during the sports activity and the physical effort, slows down gastric emptying and reduces the oxidation of ingested carbohydrates. These concomitant effects can lead to an unwanted reduction in physical performance and, in some cases, a sense of nausea.

In addition, the greater sense of satiety induced by the intake of energy bars is correlated with a lower propensity to consume additional carbohydrates. Under these conditions it is difficult for the athlete to adhere to a nutritional plan that envisages the intake of very high amounts of carbohydrates per hour, for example 90 g/h.

In addition, the time of chewing and ingestion of bars during the physical activity is longer than the time of intake of a product in liquid form. This factor can be a non-secondary disadvantage in carrying out some sports competitions.

There is therefore a need for energetic product based on carbohydrates whose formulation makes it possible to overcome or minimise the above-mentioned drawbacks.

One of the problems from which the present invention originates lies in the need to provide a source of carbohydrates to be taken during intense and/or prolonged physical activity whose composition does not slow down gastric emptying, possibly does not reduce the oxidation of ingested carbohydrates and at the same time causes a sense of satiety and satisfaction in the athlete.

Another object of the invention consists in providing an energetic product containing carbohydrates easily usable for the athlete, which is palatable and of practical use and suitable for maintaining over time high sports performance during the performance of an intense and prolonged sports activity like in cycling or endurance.

### SUMMARY OF THE INVENTION

The present invention originates from having found that by combining selected carbohydrate monomers and/or dimers in a thickened nutritional composition such as a solid gelatin, a product with a high carbohydrate content is obtained which combines the chewing properties and gustatory satisfaction typical of the bar products with the ease of intake, gastric emptying and with the nutritional advantages, such as the absence of fats and proteins typical of gels.

In accordance with a first aspect the present invention provides a product to provide the body with carbohydrates in a readily assimilable form, in particular during physical activity or a muscular effort, comprising carbohydrates selected from monosaccharides and disaccharides, water and a thickener, said product being characterized in that the monosaccharides are dextrose and fructose in a post-hydrolysis ratio of 1.6-2.2:0.8-1.2, the disaccharide is sucrose, said product being in solid form of a gelatin advantageously protein and fat free.

In accordance with a preferred embodiment the product described herein contains dextrose and fructose in a post-hydrolysis ratio of 2:1.

Preferably the product in the form of solid gelatin does not contain oligosaccharides or polysaccharides as a carbohydrate source.

Advantageously the product object of the invention is in the solid physical state and has a consistency of a gelatin.

The product object of the invention is a source of selected carbohydrates readily available and assimilable by the body of a human being.

The product is supplied in an easily chewable solid physical state suitable to meet the psychological need for chewing in the athletes engaged in a physical effort.

Advantageously, the absence of proteins and fats in the product allows to overcome the nutritional drawbacks of the conventional solid products, typically those in the form of a bar.

The product object of the invention is highly digestible but at the same time little satiating. Due to its characteristics, the product in the form of gelatin represents a suitable way to take high amounts of carbohydrates during intense and prolonged competitions and trainings and increase or maintain sports performance constant for a long period of time.

The product has a high concentration of carbohydrates, to facilitate usability and transport during sports activities.

For example, a product weighing 67 g provides approx. 40 g of carbohydrates, suitable amount to achieve the nutritional dosage up to 90 g of carbohydrates/hour in the glucose:fructose ratio described herein, preferably of 2:1.

The intake of dextrose and fructose in the ratios described herein increases the absorption of carbohydrates by the body thanks to the different mechanisms of absorption of the two monosaccharides at the intestinal level previously described. The composition of the product facilitates the intake of amounts of carbohydrates up to 90 g carbohydrates/hour as required in order to face with particularly intense and long physical efforts without a substantial drop in performance.

Preferably the product described herein is a solid sports gelatin.

Some features of embodiments of the energetic product in the form of gelatin described herein are illustrated in the enclosed Figures.

### BRIEF DESCRIPTION OF THE DRAWINGS

FIGS. 1-5 illustrate graphs showing the deformation trend, measured in accordance with the experimental tests of Example 4, of samples of the energetic product in the form of gelatin containing respectively: 3% pectin, 3.5% pectin, 4% pectin, 4.5% pectin, 5% pectin.

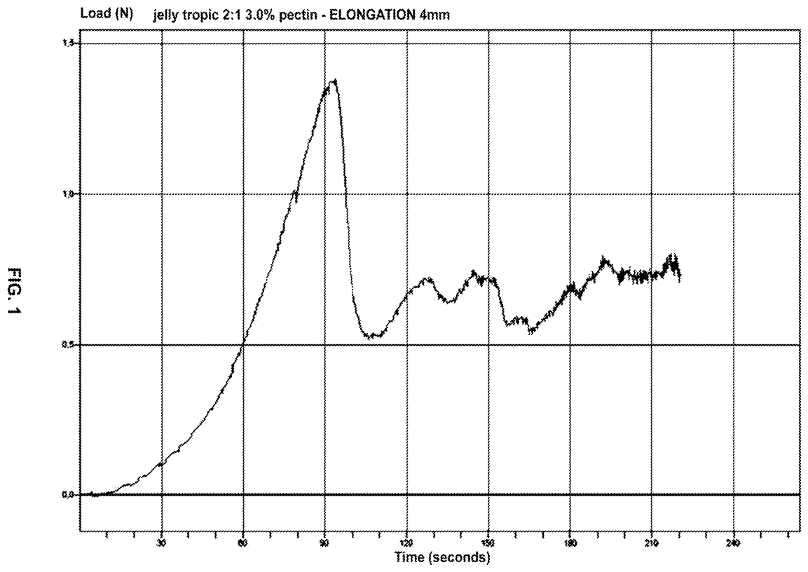

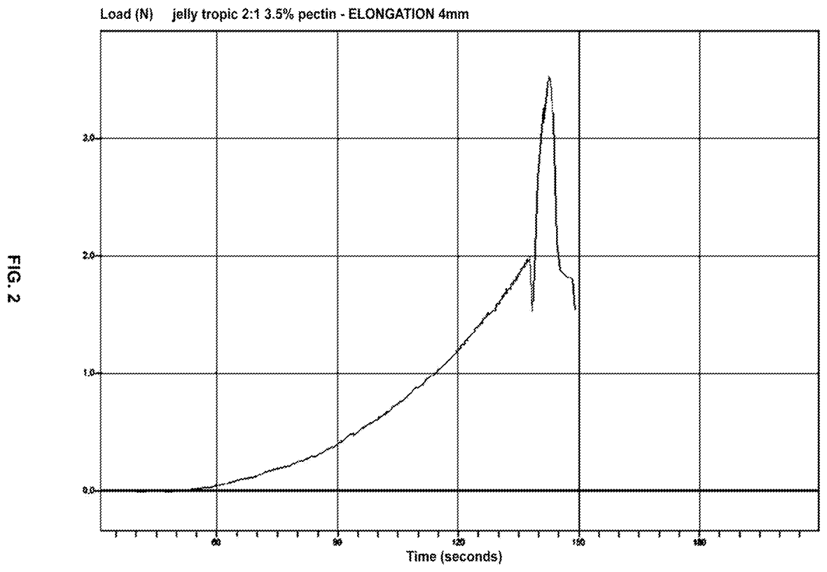

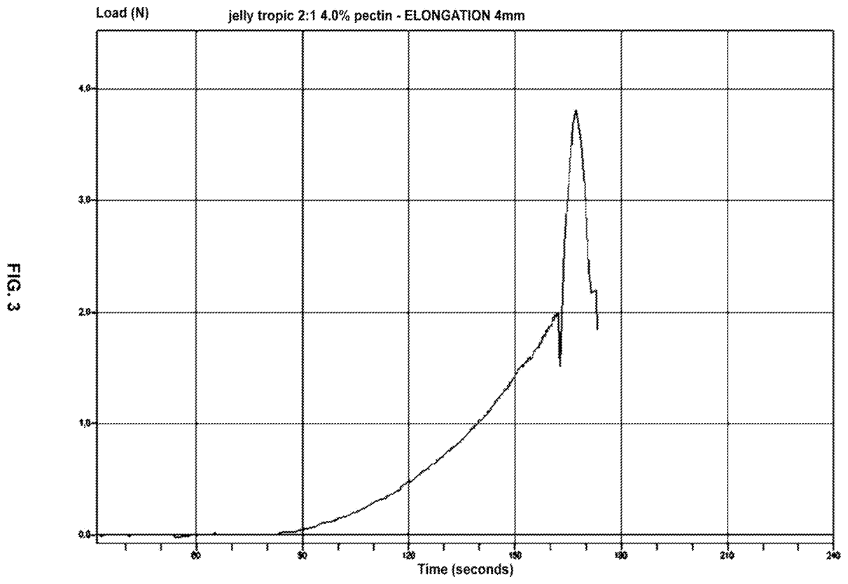

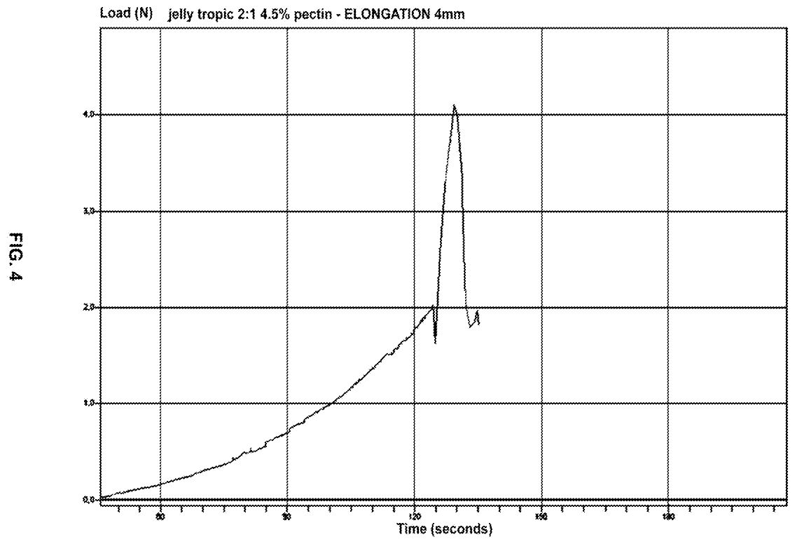

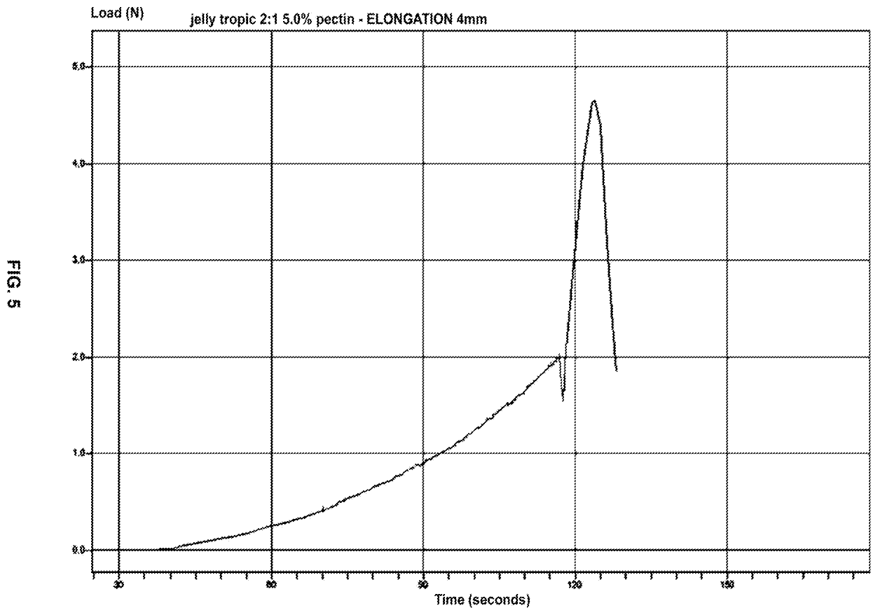

### DETAILED DESCRIPTION OF THE INVENTION

In accordance with certain aspects, the invention originates from having formulated a solid colloid product based on carbohydrates with consistency for example typical of a gelatin, comprising the monosaccharide carbohydrates dextrose and fructose and disaccharide sucrose in the ratios described herein, water and a thickener preferably pectin. Advantageously the solid colloid is free of protein material and lipids.

The specific composition of the product in carbohydrates, the water content, the presence of the thickener, combined in a suitable production process, allow obtaining a solid gelatin, extremely easy to chew and highly palatable.

The characteristics of the product solve the problems of the minimum satiating and satisfying capacity of the carbohydrate supplements in liquid or gel form and of the low digestibility of the solid products in bars also containing proteins and/or fats. In addition, the product in the form of gelatin described herein is easily chewable and ingestible in a short time.

The combination of these characteristics and properties allows to take the high amounts of carbohydrates necessary to achieve and maintain over a long period of time a high performance of the body during physical activity in which an intense physical effort is required.

Therefore, the object of the present invention is an energetic product based on carbohydrates as defined in claim 1.

Further embodiments of the invention are defined in the dependent claims 2-9.

Further objects of the invention are the gelatin suitable for sports activity defined in claim 11 and its embodiments of claims 11-14. Advantageously, the gelatin is solid. Within the scope of the present description the term gelatin is to be understood as a colloid or solid colloidal system with a typical appearance of a solid gelatin. Advantageously, gelatin is a solid, stable, homogeneous colloid.

Preferably the energetic product or gelatin suitable for the sports activity described herein is solid and has a compression breaking load of the product, expressed in Megapascal (MPa) between 0.024 MPa and 0.062 MPa, preferably between 0.046 MPa and 0.053 MPa. Advantageously, these values are measurable using an “Ametek Lloyd Instruments LS1 with hand held remote” dynamometer, using a circular punch with a diameter of 1 cm (equivalent to a 78.54 mm² test section), such as for example described in Example 4.

In accordance with preferred embodiments, the gelatin or energetic product in the form of gelatin has an amount of thickener, preferably pectin from 3 to 5% by weight and a compression breaking load of the product, expressed in Megapascal (MPa) between 0.024 MPa and 0.062 MPa, preferably between 0.046 MPa and 0.053 MPa.

These values are different from those of a “soft” bar which are typically in the range from 0.19 to 0.51 MPa of breaking load. This characteristic of the energetic product based on carbohydrates in the form of gelatin, confers the advantages above such as a greater satiating and satisfying capacity compared to carbohydrate supplements in liquid or liquid gel form and greater digestibility compared to solid products in bars also containing proteins and/or fats. In addition, the product in the form of gelatin described herein is easily chewable and ingestible in a short time.

In accordance with another aspect the present invention concerns an energetic composition or gelatin suitable for sports activity to provide the body with carbohydrates in a readily assimilable form, comprising monosaccharides and a disaccharide as a source of carbohydrates, water and a thickener, wherein
the monosaccharides are dextrose and fructose in a post-hydrolysis ratio of 1.8-2.2:0.8-1.2, preferably 1.9-2.1:0.9-1.1, the disaccharide is sucrose and
is protein and fat free.

Preferably the thickener present in the energetic gelatin is selected from pectin, starches, gelatin, a gum, for example acacia gum. The use of pectin is preferred.

The energetic gelatin may be formulated as a product in solid form in accordance with one of the embodiments described herein.

Advantageously the gelatin described herein is in solid form in a ready-to-use dosage form. For example, a single portion or dosage unit, weighing 67 g according to an embodiment described herein provides 40 g of carbohydrates, an amount easy to manage to take up an amount of carbohydrates up to 90 g/h.

One of the components of the invention consists of selected carbohydrates. Advantageously, these carbohydrates are in the form of monomers (monosaccharides) and/or dimers (disaccharides).

Advantageously carbohydrates oligomers and/or polymers are absent in the energetic product or gelatin.

A selected monomeric carbohydrate (monosaccharide) contained in the product is the dextrose. This simple sugar is quickly absorbed and oxidized during the physical exercise. In addition, it allows obtaining a texture that is easy to chew, not excessively sweet and less compact.

These characteristics are not obtained with glucose syrups, which, although having a discrete dextrose content, also contain several glucidic species, such as dimers and oligomers (polysaccharides).

In accordance with certain embodiments the energetic product or gelatin described herein has a dextrose content of at least 20%, preferably greater than 30%, in particular in the range from 30 to 40% by weight, for example equal to 35%.

Another selected monomeric carbohydrate contained in the product is the fructose. In accordance with some embodiments this monosaccharide is present as fructose syrup.

This monosaccharide is important in order to obtain a glucose:fructose post-hydrolysis ratio of the sugars of 1.6-2.2:0.8-1.2, and thus maximize the transport of monomeric carbohydrates through intestinal transporters.

Another carbohydrate contained in the formulation of the invention is a disaccharide, in particular the sucrose. The presence of this disaccharide contributes to the solid physical form of gelatin/colloid.

The disaccharide sucrose was also selected due to the high glycemic index and ease of digestion and for the supply of glucose and fructose monosaccharides necessary to obtain a glucose:fructose ratio in a post-hydrolysis ratio of 1.6-2.2:0.8-1.2.

In accordance with some embodiments the energetic product or energetic gelatin contains sucrose in an amount in the range from 15% to 20%, in particular 16.5% by weight.

Preferably the ratio between carbohydrate monomers (monosaccharides) and dimers (disaccharides) to obtain the product in gelatin/solid colloid form is greater than 2, in particular from 2.5 to 3.1, preferably 2.8.

According to some embodiments the product contains a thickener, preferably pectin, in an amount from 3 to 5% by weight, in particular from 3.5 to 4.5%, for example 4%, to obtain the chewable, non-sticky, solid colloid/gelatin form.

In accordance with some embodiments the product contains water in an amount in the range from 25% to 35%, for example 31%.

Preferably the product has a carbohydrate content greater than 50% by weight, for example of 60%.

Advantageously, the combination of glucose and fructose in the selected ratios of the composition, allows to reach a higher rate of carbohydrate oxidation than it can be reached by taking only glucose as an energy source, in particular in the nutritional programs that envisage carbohydrate intakes greater than 60 g/h.

The combined intake of the monosaccharides glucose and fructose present in the product described increases the absorption of carbohydrates thanks to the diversity of the absorption mechanisms of the two monosaccharides at the intestinal level.

This mechanism contributes to accelerating the intestinal absorption of the carbohydrates by increasing the availability of exogenous carbohydrates in the blood.

Although the intestinal absorption of the carbohydrates limits the intestinal absorption of glucose by the SGLT1 transporter to about 1 g/min, the intake of glucose in combination with fructose as described herein increases the rate of carbohydrate absorption and oxidation to at least 1.5 g/min since the fructose uses the GLUT5 transporter which is different from that of the glucose.

Advantageously, in order to achieve a carbohydrate assimilation rate of 80-90 g/hour and increase the sports performance and/or the muscle endurance, the weight ratio glucose from 1.6 to 2.2:fructose from 0.8 to 1.2; preferably glucose 1.8-2.2 fructose 0.8-1.2; even more preferably glucose 1.9-2.1:fructose 0.9-1.1, for example 2:1, according to certain aspects of the invention, contributes significantly to increasing carbohydrate assimilability and gastric tolerance.

The composition of the product or gelatin as defined herein also solves the problem of the competitive intestinal assimilation of the monosaccharides and of the possibility of intake of a carbohydrate source by an individual who is performing an aerobic sports activity, such as jogging, endurance, cycling, running or marathon.

In accordance with another aspect the present invention provides the use of an energetic product based on carbohydrates according to any of the embodiments described herein to support and/or improve the physical or sports performance and/or reduce muscle fatigue during a physical exercise, particularly at high intensity and duration, preferably from 30 minutes to 8 hours, from 1 hour to 4 hours, from one hour and thirty minutes to two hours and thirty minutes, e.g. 2 hours.

The advantages of the product object of the invention are more evident considering that according to the most recent guidelines of sports medicine, the intake of carbohydrates during intense sports activity is generally structured as follows:
for activities lasting around 2 hours, it is recommended to take quantities of carbohydrates up to 60 g carbohydrates/hour;
in the case of particularly intense and long physical efforts, for example exceeding 2.5-3 hours, the supply can reach up to 90 g of carbohydrates/hour.

When the sports performance requires a carbohydrate supply of 90 g/h, the problems referable to the competitive absorption of the different types of carbohydrates arise.

Under these conditions, the glucose/fructose ratio described herein plays a very important role for the purposes of achieving sports performance. The more this ratio approaches the post-hydrolysis glucose:fructose ratio of carbohydrates of 1.8-2.2:0.8-1.2, preferably 1.9-2.1:0.9-1.1, the greater the chances of increasing carbohydrate absorption and of achieving an increase in sports performance are. The energetic product may also contain additional ingredients in addition to the glucidic component.

According to some embodiments the product described herein comprises one or more biologically active substances such as for example caffeine, carnitine, coenzyme Q10 and their mixtures.

In accordance with some embodiments the product described herein further contains at least one vitamin e.g. one or more fat-soluble vitamins such as vitamin A, D and/or water-soluble vitamins such as vitamin C, vitamins belonging to group B, such as vitamin B1, B2, B6, B12 and their mixtures.

Preferably the product contains vitamin B1 as it contributes to the physiological functioning of saccharide metabolism and to ribose synthesis. In addition, as a coenzyme thiamine pyrophosphate it intervenes in the removal of CO₂ associated with oxidation and fatty acid synthesis.

In some embodiments the product herein may further contain minerals such as Potassium, Magnesium, Zinc etc.

Food additives such as preservatives, flavourings, acidity regulators, colourings may also be present in the product.

In some embodiments the energetic product or gelatin described herein may also contain other disaccharides, for example trehalose.

In accordance with one aspect of the invention the energetic product is a food or dietary supplement, a nutritional or enriched foodstuff whose use is indicated to the population of subjects practicing sports activities in particular of a motor type such as jogging, marathon, endurance and in general any type of endurance running or cycling activity.

Within the scope of the present description the term composition may assume the meaning of gelatin and in that case the two terms may be interchangeable.

The following examples are provided primarily to illustrate the present invention and do not intend to limit the scope of protection as is apparent from the appended claims.

### Example 1

### Supplement in Solid Form of Chewable Gelatin

Dextrose: 35%.
Water: 26%
Fructose syrup: 17%
Sucrose: 16.5%
Thickener (e.g. pectin): 4%
Vitamin B1 15% NRV

### Example 2

Energetic product in the form of solid gelatin with a high concentration of carbohydrates containing
25 Dextrose: 35%.
Water: 26%
Fructose syrup: 17%
Sucrose: 16.5%
Pectin: 4.5%

### Example 3

### Process of Production of the Energetic Product in the Form of Solid Gelatin.

The following is a diagram of the process of production of an energetic product according to Examples 1-2.

The process comprises the following steps:
hot premixing of water and ingredients, in particular the monosaccharide carbohydrates dextrose, fructose and disaccharides sucrose,
adding the thickener in particular pectin;
activation of the pectin with an acidic solution of citric acid (preferably with a citric acid content of 25%) and mixing;
pumping in the bags and packaging.
The packaged product is stored at room temperature to allow gelification.
Subsequently, secondary packaging and palletizing takes place.

### Example 4

Compression breaking load test on samples of energetic product in the form of gelatin with amounts of thickener from 3 to 5% by weight.

The deformation trend of the energetic product in the form of solid gelatin was measured experimentally using the 4 mm punch elongation protocol as described herein.

5 samples of energetic product in the form of solid gelatin were used in the tests.

### Materials and Methods

An “Ametek Lloyd Instruments LS1 with hand held remote” dynamometer was used for the analyses, using a circular punch with a diameter of 1 cm (section 78.54 mm²). The force was delivered from the top downwards, in a unilateral compression regime.

Different elongations of the punch (3, 4, 5, 7 mm) were used for greater reproducibility of the results.

Product samples with the following pectin contents were analysed: 3% pectin, 3.5% pectin, 4% pectin, 4.5% pectin, 5% pectin. The results obtained in the form of a graph are illustrated in the enclosed figures.

All samples tested had a pectin content comprised from 3% to 5% pectin by weight. The preferable results are referable to the samples with pectin content comprised from 3.5% to 4.5% by weight.

### Results

The analysis made it possible to identify the following results:
compression breaking load of the product (expressed in Megapascal, MPa);
graph showing the trend of the product to deformation.

The results found, illustrated in the graphs of the enclosed figures, show the trend of the product to deformation with the 4 mm punch elongation protocol.

The calculation results are given in the following Table.

| PECTIN TO FINAL | PUNCH | MAX FORCE | MAX FORCE | | BREAKING | | BREAKING | |
|---|---|---|---|---|---|---|---|---|
| PRODUCT | ELONGATION | DEFORMATION | DEFORMATION | | LOAD | | LOAD | |
| PERCENTAGE (%) | (mm) | (N) | (N) - MEAN | | (mPa) | | (mPa) - MEAN | PECTIN % |
| jelly tropic 2:1 | 4.0000 mm | 4.6527 | | | | 0.0592 | | |
| 5.0% pectin | | | | | | | | |
| jelly tropic 2:1 | 4.0000 mm | 4.6517 | | | | 0.0592 | | |
| 5.0% pectin | | | | | | | | |
| jelly tropic 2:1 | 3.0000 mm | 4.7754 | | | | 0.0608 | | |
| 5.0% pectin | | | | | | | | |
| jelly tropic 2:1 | 5.0000 mm | 5.1625 | | | | 0.0657 | | |
| 5.0% pectin | | | | | | | | |
| jelly tropic 2:1 | 7.0000 mm | 5.2387 | [symbol] | 4.90 | [symbol] | 0.0667 | 0.062 | 5.0 |
| 5.0% pectin | | | | | | | | |
| jelly tropic 2:1 | 4.0000 mm | 4.1047 | | | | 0.0523 | | |
| 4.5% pectin | | | | | | | | |
| jelly trapic 2:1 | 4.0000 mm | 4.5548 | | | | 0.0580 | | |
| 4.5% pectin | | | | | | | | |
| jelly tropic 2:1 | 3.0000 mm | 3.584 | | | | 0.0456 | | |
| 4.5% pectin | | | | | | | | |
| jelly tropic 2:1 | 5.0000 mm | 3.8379 | | | | 0.0489 | | |
| 4.5% pectin | | | | | | | | |
| jelly tropic 2:1 | 7.0000 mm | 3.9625 | [symbol] | 4.01 | [symbol] | 0.0505 | 0.051 | 4.5 |
| 4.5% pectin | | | | | | | | |
| jelly tropic 2:1 | 4.0000 mm | 3.8161 | | | | 0.0486 | | |
| 4.0% pectin | | | | | | | | |
| jelly tropic 2:1 | 4.0000 mm | 3.954 | | | | 0.0503 | | |
| 4.0% pectin | | | | | | | | |
| jelly tropic 2:1 | 3.0000 mm | 4.232 | | | | 0.0539 | | |
| 4.0% pectin | | | | | | | | |
| jelly tropic 2:1 | 5.0000 mm | 4.5217 | | | | 0.0576 | | |
| 4.0% pectin | | | | | | | | |
| jelly tropic 2:1 | 7.0000 mm | 4.3551 | [symbol] | 4.18 | [symbol] | 0.0555 | 0.053 | 4.0 |
| 4.0% pectin | | | | | | | | |
| jelly tropic 2:1 | 4.0000 mm | 3.5317 | | | | 0.0450 | | |
| 3.5% pectin | | | | | | | | |
| jelly tropic 2:1 | 4.0000 mm | 3.5642 | | | | 0.0454 | | |
| 3.5% pectin | | | | | | | | |
| jelly tropic 2:1 | 3.0000 mm | 3.8014 | | | | 0.0484 | | |
| 3.5% pectin | | | | | | | | |
| jelly tropic 2:1 | 5.0000 mm | 3.4818 | | | | 0.0443 | | |
| 3.5% pectin | | | | | | | | |
| jelly tropic 2:1 | 7.0000 mm | 3.6373 | [symbol] | 3.60 | [symbol] | 0.0463 | 0.046 | 3.5 |
| 3.5% pectin | | | | | | | | |
| jelly tropic 2:1 | 4.0000 mm | 2.6086 | | | | 0.0332 | | |
| 3.0% pectin | | | | | | | | |
| jelly tropic 2:1 | 4.0000 mm | 1.3855 | | | | 0.0176 | | |
| 3.0% pectin | | | | | | | | |
| jelly tropic 2:1 | 3.0000 mm | 1.6225 | | | | 0.0207 | | |
| 3.0% pectin | | | | | | | | |
| jelly tropic 2:1 | 3.0000 mm | 1.8583 | | | | 0.0237 | | |
| 3.0% pectin | | | | | | | | |
| jelly tropic 2:1 | 3.0000 mm | 1.8719 | [symbol] | 1.87 | [symbol] | 0.0238 | 0.024 | 3.0 |
| 3.0% pectin | | | | | | | | |

1. Energetic product based on carbohydrates to provide the body with carbohydrates in a readily assimilable form during physical activity or muscular effort, wherein said product comprises carbohydrates selected from monosaccharides and disaccharides, water and a thickener and wherein the monosaccharides are dextrose and fructose in a post-hydrolysis ratio of 1.6-2.2:0.8-1.2, the disaccharide is sucrose, and wherein said product is in solid form of a gelatin which is protein and fat free.

2. Energetic product according to claim 1, wherein the thickener is selected from pectin, starches, gelatin and acacia gum.

3. Energetic product according to claim 1, wherein said product it contains the monosaccharides dextrose and fructose in a post-hydrolysis ratio of 1.9-2.1:0.9-1.1.

4. The energetic product according to claim 1, wherein said product contains dextrose in an amount of at least 20% by weight.

5. Energetic product according to claim 1, wherein said product contains sucrose in an amount comprised from 15% to 18% by weight.

6. Energetic product according to claim 1, wherein said product contains fructose in an amount from 10 to 20% by weight.

7. Energetic product according to claim 1, further comprising one or more fat-soluble vitamins, and/or water-soluble vitamins, vitamins belonging to group B and their mixtures.

8. Energetic product according to claim 1, further comprising a biologically active substance, coenzyme Q10 and their mixtures and/or mineral salts-preferably selected from potassium, magnesium and sodium salts.

9. Energetic product according to claim 1, wherein said product is in the form of solid gelatin having a compression breaking load, expressed in Megapascal (MPa), comprised from 0.024 MPa to 0.062 MPa.

10. A method for supporting and/or improving physical performance and/or reducing muscle fatigue during physical exercise, at high density and long duration, comprising the steps of
eating the energetic product according to claim 1.

11. A chewable gelatin to be taken during sports activity to provide the body with carbohydrates in a readily assimilable form, comprising monosaccharides and a disaccharide as a source of carbohydrates, water and a thickener, wherein the monosaccharides are dextrose and fructose in a post-hydrolysis ratio of 1.8-2.2:0.8-1.2, and the disaccharide is sucrose.

12. The chewable gelatin according to claim 11, wherein said chewable gelatin it is a solid gelatin.

13. The chewable gelatin according to claim 11, wherein said chewable gelatin is protein and fat free.

14. The chewable gelatin according to claim 11, wherein said chewable gelatin is in the form of solid gelatin having a maximum deformation load, expressed in Megapascal (MPa), comprised from 0.024 MPa to 0.062 MPa.

15. A method for supporting and/or improving physical performance and/or reducing muscle fatigue during physical exercise at high density and long duration, said method comprising a step of eating the chewable gelatin according to claim 11.
